(19) United States
(12) Patent Application Publication
(10) Pub. No.: US 2022/0409577 A1
(43) Pub. Date: Dec. 29, 2022

(54) INTRATUMORAL ADMINISTRATION OF ASCORBIC ACID AND QUINONE COMPOUNDS FOR TUMOR TREATMENT

(71) Applicant: IC-MedTech Corp., Las Vegas, NV (US)

(72) Inventors: Thomas M. Miller, Las Vegas, NV (US); Zhivko Zhelev, Sofia (BG)

(21) Appl. No.: 17/806,695

(22) Filed: Jun. 13, 2022

Related U.S. Application Data
(60) Provisional application No. 63/210,399, filed on Jun. 14, 2021.

(51) Int. Cl. A61K 31/375; A61K 31/122; A61P 35/00; A61P 25/00

(52) U.S. Cl. CPC A61K 31/375; A61K 31/122; A61P 35/00; A61P 25/00

(57) ABSTRACT

Provided herein is a method of treating or alleviating one or more symptoms of a tumor in a subject, comprising intratumorally administering to the subject a therapeutically effective amount of ascorbic acid and a quinone compound

### CROSS REFERENCE TO RELATED APPLICATION

This application claims the benefit of the priority of U.S. Provisional Application No. 63/210,399, filed Jun. 14, 2021; the disclosure of which is incorporated herein by reference in its entirety.

### FIELD

Provided herein is a method of treating or alleviating one or more symptoms of a tumor in a subject, comprising intratumorally administering to the subject a therapeutically effective amount of ascorbic acid and a quinone compound.

### BACKGROUND

Brain tumors are generally classified into primary and secondary brain tumors. Alexandru et al., Prog. Neurol. Surg. 2012, 25, 13-29; Vargo, Phys. Med. Rehabil. Clin. N. Am. 2017, 28, 115-41. Primary brain tumors originate from the brain or the brain's immediate surroundings. Perkins and Liu, Am. Fam. Physician. 2016, 93, 211-17. Secondary brain tumors, also known as metastatic brain tumors, start elsewhere in the body and spread to the brain. Lah et al.. Semin. Cancer Biol. 2020, 60, 262-73. Secondary brain tumors are about four times as common as primary brain tumors, with about half of metastases migrating from lung cancer. Alexandru et al., Prog. Neurol. Surg. 2012, 25, 13-29; Lah et al., Semin. Cancer Biol. 2020, 60, 262-73. Typical symptoms of a brain tumor include headaches, nausea, vomiting, seizures, neurocognitive symptoms, and personality changes. Chandana et al., Am. Fam. Physician. 2008, 77, 1423-30; Perkins and Liu, Am. Fam. Physician. 2016, 93, 211-17.

Gliomas are the most prevalent type of adult brain tumors, accounting for over 75 percent of malignant brain tumors. Chaichana et al., Ther. Deliv. 2015, 6, 353-69; Ostrom et al., Nuero-Oncol. 2016, 18. v1-v75. Glioblastoma multiforme (GBM), the highest-grade glioma, is the deadliest form of brain tumors, with a median survival rate of less than 2 years after diagnosis and an average 5-year survival rate of less than 5%. Batash et al., Curr. Med. Chem. 2017, 24, 3002-9; Jackson et al., Nat. Immunol. 2019, 20, 1100-9. Brain tumors are also the most common and most lethal of all pediatric solid tumors. Smith & Reaman, Pediatr. Clin. North Am. 2015, 62, 301-12. Despite the advances in cancer treatment, brain tumors remain among the deadliest of all forms of cancer, with no new, more effective therapies being developed in the past 30 years. Batash et al., Curr. Med. Chem. 2017, 24, 3002-9; Aldape et al., Nat. Rev. Clin. Oncol. 2019, 16, 509-20; Gedeon et al., Expert Rev. Clin. Pharmacol. 2020, 13, 1147-58. Therefore, there is an urgent need for an effective therapy for a brain tumor.

### SUMMARY OF THE DISCLOSURE

Provided herein is a method of treating or alleviating one or more symptoms of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

Also provided herein is a method of inhibiting the growth of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

Additionally, provided herein is a method of increasing the quality of life of a subject having a tumor, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

Furthermore, provided herein is a method of inhibiting the growth of a tumorous cell, comprising contacting the tumorous cell with an effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

### BRIEF DESCRIPTION OF THE DRAWINGS

FIG. 1 shows the antitumor effect of vitamin C and menadione against glioblastoma in a xenograft mouse model (GS9L), where vitamin C and menadione were administered intracranially once on Day 4 (i.e., 3 days after the implantation of glioblastoma cells).

FIG. 2 shows a survival curve of glioblastoma mice in a xenograft mouse model (GS9L), where vitamin C and menadione were administered intracranially once on Day 4 (i.e., 3 days after the implantation of glioblastoma cells).

FIG. 3 shows the antitumor effect of vitamin C and menadione against glioblastoma in a xenograft mouse model (U87MG), where vitamin C and menadione were administered intracranially once on Day 7 (i.e., 6 days after the implantation of glioblastoma cells).

FIG. 4 shows a survival curve of glioblastoma mice in a xenograft mouse model (U87MG), where vitamin C and menadione were administered intracranially once on Day 7 (i.e., 6 days after the implantation of glioblastoma cells).

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), livestock, a domestic pet, cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat.” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The terms “therapeutically effective amount” and “effective amount” are meant to include the amount of a compound or a combination of compounds that, when administered, is sufficient to prevent development of, or alleviate to some extent, one or more of the symptoms of the disorder, disease, or condition being treated. The term “therapeutically effective amount” or “effective amount” also refers to the amount of a compound or a combination of compounds that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer of a compound described herein.

The term “quality of life” or “QOL” refers to a maintenance or increase in a the overall physical and mental state of a subject, for example, a subject with a glioma, such as the subject's cognitive ability, ability to communicate and interact with others, decreased dependence upon analgesics for pain control, maintenance of ambulatory ability, maintenance of appetite and body weight (lack of cachexia), lack of or diminished feeling of “hopelessness;” continued interest in playing a role in treatment, and other similar mental and physical states.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]-pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]-pyridinyl, and imidazo[4,5-c]pyridinyl), imidazothiazolyl (including all isomeric forms, e.g., imidazo[2,1-b]-thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thia-diazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]-pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, alkenyl, alkynyl, cycloalkyl, aryl, aralkyl, heteroaryl, or heterocyclyl group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NRd)NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R⁶, —NR^{e}C(O)OR^{f}, —NR^{e}C(O)NR^{f}R^{g}, —NR^{e}C(O)SR^{f}, —NR^{e}C(NR⁶)NR^{f}R^{g}, —NR^{e}C(S)R⁶, —NR^{e}C(S)OR^{f}, —NR^{e}C(S)NR^{f}R^{g}, —NR^{e}S(O)R⁶, —NR^{e}S(O)₂Rh, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R⁶ is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (⁶Cl), chlorine-37 (³⁷Cl), bromine-79 (⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

### Ascorbic Acid Compound

In one embodiment, the ascorbic acid compound is L-ascorbic acid or a pharmaceutically acceptable salt thereof; or a pharmaceutically acceptable solvate or hydrate thereof. In another embodiment, the ascorbic acid compound is vitamin C, L-xyloascorbic acid, 3-oxo-L-gulofuranolactone (enol form), L-3-ketothreohexuronic acid lactone, antiscorbutic vitamin, cevitamic acid, adenex, allercorb, ascorin, ascorteal, ascorvit, cantan, cantaxin, catavin C, cebicure, cebion, cecon, cegiolan, celaskon, celin, cenetone, cereon, cergona, cescorbat, cetamid, cetabe, cetemican, cevalin, cevatine, cevex, cevimin, ce-vi-sol, cevitan, cevitex, cewin, ciamin, cipca, concemin, C-vin, daviamon C, duoscorb, hybrin, laroscorbine, lemascorb, planavit C, proscorbin, redoxon, ribena, scorbacid, scorbu-C, testascorbic, vicelat, vitacee, vitacimin, vitacin, vitascorbol, or xitix. In yet another embodiment, the ascorbic acid compound is L-ascorbic acid. In still another embodiment, the ascorbic acid compound is a pharmaceutically acceptable salt of L-ascorbic acid, or a pharmaceutically acceptable solvate or hydrate thereof.

Suitable bases for forming a pharmaceutically acceptable salt of L-ascorbic acid include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole. L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine. N-methyl-D-glucamine, 2-amino-2-(hydroxymethyl)-1,3-propanediol, and tromethamine.

In one embodiment, the ascorbic acid compound is an alkali or alkaline earth metal salt of L-ascorbic acid, or a pharmaceutically acceptable solvate or hydrate thereof. In another embodiment, the ascorbic acid compound is sodium, potassium, calcium, or magnesium L-ascorbate, or a pharmaceutically acceptable solvate or hydrate thereof. In yet another embodiment, the ascorbic acid compound is sodium L-ascorbate, or a pharmaceutically acceptable solvate or hydrate thereof. In yet another embodiment, the ascorbic acid compound is sodium L-ascorbate. In yet another embodiment, the ascorbic acid compound is vitamin C sodium, ascorbin, sodascorbate, natrascorb, cenolate, ascorbicin, or cebitate. In yet another embodiment, the ascorbic acid compound is potassium L-ascorbate, or a pharmaceutically acceptable solvate or hydrate thereof. In yet another embodiment, the ascorbic acid compound is calcium L-ascorbate, or a pharmaceutically acceptable solvate or hydrate thereof. In yet another embodiment, the ascorbic acid compound is calcium L-ascorbate. In yet another embodiment, the ascorbic acid compound is magnesium L-ascorbate, or a pharmaceutically acceptable solvate or hydrate thereof. In still another embodiment, the ascorbic acid compound is magnesium L-ascorbate.

In certain embodiments, the ascorbic acid compound is D-ascorbic acid or a pharmaceutically acceptable salt, or a pharmaceutically acceptable solvate or hydrate thereof.

In one embodiment, the ascorbic acid compound is D-ascorbic acid. In another embodiment, the ascorbic acid compound is a pharmaceutically acceptable salt of D-ascorbic acid, or a pharmaceutically acceptable solvate or hydrate thereof.

Suitable bases for forming a pharmaceutically acceptable salt of D-ascorbic acid include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole. L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

In one embodiment, the ascorbic acid compound is an alkali or alkaline earth metal salt of D-ascorbic acid, or a pharmaceutically acceptable solvate or hydrate thereof. In another embodiment, the ascorbic acid compound is sodium, potassium, calcium, or magnesium D-ascorbate, or a pharmaceutically acceptable solvate or hydrate thereof. In yet another embodiment, the ascorbic acid compound is sodium D-ascorbate, or a pharmaceutically acceptable solvate or hydrate thereof. In yet another embodiment, the ascorbic acid compound is sodium D-ascorbate. In yet another embodiment, the ascorbic acid compound is potassium D-ascorbate, or a pharmaceutically acceptable solvate or hydrate thereof. In yet another embodiment, the ascorbic acid compound is calcium D-ascorbate, or a pharmaceutically acceptable solvate or hydrate thereof. In yet another embodiment, the ascorbic acid compound is calcium D-ascorbate. In yet another embodiment, the ascorbic acid compound is magnesium D-ascorbate, or a pharmaceutically acceptable solvate or hydrate thereof. In still another embodiment, the ascorbic acid compound is magnesium D-ascorbate.

### Quinone Compounds

In one embodiment, the quinone compound is vitamin K. In certain embodiments, the vitamin K is a 2-methyl-1,4-naphthoquinone of Formula (I), (II), or (III):

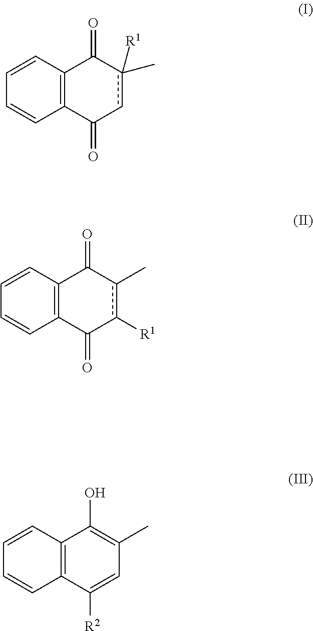

or an enantiomer, a mixture of enantiomers, or a mixture of two or more diastereomers thereof; or a pharmaceutically acceptable salt, solvate, or hydrate thereof; wherein R¹ is hydrogen, C₁20 alkyl, C₂₋₂₀ alkenyl, C₂₋₂₀ alkynyl, or —SO₃H; and R² is hydroxyl or amino.

In certain embodiments, the vitamin K is vitamin K₁, vitamin K₂, vitamin K₃, vitamin K₄, vitamin K₅, or a mixture of two or more thereof.

In one embodiment, the vitamin K is vitamin K₁, or a pharmaceutically acceptable salt, solvate, or hydrate thereof. In one embodiment, vitamin K₁ is phylloquinone, [R—[R*,R*-(E)]]-2-methyl-3-(3,7,11,15-tetramethyl-2-hexadecenyl)-1,4-naphthalenedione, 2-methyl-3-phytyl-1,4-naphthoquinone, 3-phytylmenadione, phytomenadione, phytonadione, aqua-merphyton, konakion, mephyton, mono-day, veda-K₁, or veta-K₁.

In another embodiment, the vitamin K is vitamin K₂, or a pharmaceutically acceptable salt, solvate, or hydrate thereof. In one embodiment, vitamin K₂ is a menaquinone or 2-methyl-3-all-trans-polyprenyl-1,4-naphthoquinone. In another embodiment, vitamin K₂ is menaquinone 4, which is also known as vitamin K₂₍₂₀₎; menaquinone 6, which is also known as vitamin K₂₍₃₀₎; or menaquinone 7, which is also known as vitamin K₂₍₃₅₎.

In yet another embodiment, the vitamin K is vitamin K₃, or a pharmaceutically acceptable salt, solvate, or hydrate thereof. In one embodiment, vitamin K₃ is menadione, 2-methyl-1,4-naphthalenedione, 2-methyl-1,4-naphthoquinone, menaphthone, vitamin K₂₍₀₎, kanone, kappaxin, kayklot, kayquinone, klottone, kolklot, thyloquinone, 1,2,3,4-tetrahydro-2-methyl-1,4-dioxo-2-naphthalenesulfonic acid, or sodium 1,2,3,4-tetrahydro-2-methyl-1,4-dioxo-2-naphthalenesulfonate. In another embodiment, vitamin K₃ is menadione (i.e., 2-methyl-1,4-naphthalenedione).

In one embodiment, vitamin K₃ is 1,2,3,4-tetrahydro-2-methyl-1,4-dioxo-2-naphthalenesulfonic acid, or a pharmaceutically acceptable salt, solvate, or hydrate thereof. In another embodiment, vitamin K₃ is 1,2,3,4-tetrahydro-2-methyl-1,4-dioxo-2-naphthalenesulfonate (also known as menadione bisulfite), or a pharmaceutically acceptable solvate or hydrate thereof. Suitable bases for forming a pharmaceutically acceptable salt include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole. L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine. 2-amino-2-(hydroxymethyl)-1,3-propanediol, and tromethamine.

In one embodiment, vitamin K₃ is an alkali or alkaline earth metal salt of 1,2,3,4-tetrahydro-2-methyl-1,4-dioxo-2-naphthalenesulfonic acid, or a pharmaceutically acceptable solvate or hydrate thereof. In another embodiment, vitamin K₃ is sodium, potassium, calcium, or magnesium 1,2,3,4-tetrahydro-2-methyl-1,4-dioxo-2-naphthalenesulfonate, or a pharmaceutically acceptable solvate or hydrate thereof. In yet another embodiment, vitamin K₃ is sodium 1,2,3,4-tetrahydro-2-methyl-1,4-dioxo-2-naphthalenesulfonate, or a pharmaceutically acceptable solvate or hydrate thereof. In yet another embodiment, vitamin K₃ is potassium 1,2,3,4-tetrahydro-2-methyl-1,4-dioxo-2-naphthalenesulfonate, or a pharmaceutically acceptable solvate or hydrate thereof. In yet another embodiment, vitamin K₃ is magnesium 1,2,3,4-tetrahydro-2-methyl-1,4-dioxo-2-naphthalenesulfonate, or a pharmaceutically acceptable solvate or hydrate thereof. In yet another embodiment, vitamin K₃ is sodium 1,2,3,4-tetrahydro-2-methyl-1,4-dioxo-2-naphthalenesulfonate. In yet another embodiment, vitamin K₃ is anhydrous sodium 1,2,3,4-tetrahydro-2-methyl-1,4-dioxo-2-naphthalenesulfonate. In yet another embodiment, vitamin K₃ is sodium 1,2,3,4-tetrahydro-2-methyl-1,4-dioxo-2-naphthalenesulfonate hydrate. In still another embodiment, vitamin K₃ is sodium 1,2,3,4-tetrahydro-2-methyl-1,4-dioxo-2-naphthalenesulfonate trihydrate.

In yet another embodiment, the vitamin K is vitamin K₄, or a pharmaceutically acceptable salt, solvate, or hydrate thereof. In one embodiment, vitamin K₄ is menadiol, 2-methyl-1,4-naphthalenediol, 2-methyl-1,4-naphthohydroquinone, 2-methyl-1,4-naphthoquinol, or dihydrovitamin K₃. In another embodiment, vitamin K₄ is menadiol sodium diphosphate.

In certain embodiments, the vitamin K comprises vitamins K₃ and K₄, or pharmaceutically acceptable salts, solvates, or hydrates thereof.

In still another embodiment, the vitamin K is vitamin K₅, or a pharmaceutically acceptable salt, solvate, or hydrate thereof. In one embodiment, vitamin K₅ is 4-amino-2-methyl-1-naphthalenol, 4-amino-2-methyl-1-naphthol, 1-hydroxy-2-methyl-4-aminonaphalene, 2-methyl-4-amino-1-hydroxynaphthalene, 2-methyl-4-amino-1-naphthol, 3-methyl-4-hydroxy-1-naphthylamine, or synkamin.

In certain embodiments, the vitamin K is chromium-free. In certain embodiments, the chromium-free vitamin K contains no more than about 100 ppm, about 50 ppm, about 20 ppm, about 10 ppm, about 5 ppm, about 2 ppm, about 1 ppm, about 0.1 ppm, about 10 ppb, or about 1 ppb of chromium. In certain embodiments, the chromium-free vitamin K contains no more than about 100 ppm of chromium. In certain embodiments, the chromium-free vitamin K contains no more than about 50 ppm of chromium. In certain embodiments, the chromium-free vitamin K contains no more than about 20 ppm of chromium. In certain embodiments, the chromium-free vitamin K contains no more than about 10 ppm of chromium. In certain embodiments, the chromium-free vitamin K contains no more than about 5 ppm of chromium. In certain embodiments, the chromium-free vitamin K contains no more than about 2 ppm of chromium. In certain embodiments, the chromium-free vitamin K contains no more than about 1 ppm of chromium. In certain embodiments, the chromium-free vitamin K contains no more than about 0.1 ppm of chromium. In certain embodiments, the chromium-free vitamin K contains no more than about 10 ppb of chromium. In certain embodiments, the chromium-free vitamin K contains no more than about 1 ppb of chromium.

In certain embodiments, the vitamin K is chromium-free vitamin K₃. In certain embodiments, the chromium-free vitamin K₃ contains no more than about 100 ppm, about 50 ppm, about 20 ppm, about 10 ppm, about 5 ppm, about 2 ppm, about 1 ppm, about 0.1 ppm, about 10 ppb, or about 1 ppb of chromium. In certain embodiments, the chromium-free vitamin K₃ contains no greater than about 100 ppm of chromium. In certain embodiments, the chromium-free vitamin K₃ contains no greater than about 50 ppm of chromium. In certain embodiments, the chromium-free vitamin K₃ contains no greater than about 20 ppm of chromium. In certain embodiments, the chromium-free vitamin K₃ contains no greater than about 10 ppm of chromium. In certain embodiments, the chromium-free vitamin K₃ contains no greater than about 5 ppm of chromium. In certain embodiments, the chromium-free vitamin K₃ contains no greater than about 2 ppm of chromium. In certain embodiments, the chromium-free vitamin K₃ contains no greater than about 1 ppm of chromium. In certain embodiments, the chromium-free vitamin K₃ contains no greater than about 0.1 ppm of chromium. In certain embodiments, the chromium-free vitamin K₃ contains no greater than about 10 ppb of chromium. In certain embodiments, the chromium-free vitamin K₃ contains no greater than about 1 ppb of chromium.

In certain embodiments, the vitamin K is chromium-free sodium 1,2,3,4-tetrahydro-2-methyl-1,4-dioxo-2-naphthalenesulfonate. In certain embodiments, the chromium-free sodium 1,2,3,4-tetrahydro-2-methyl-1,4-dioxo-2-naphthalenesulfonate contains no more than about 100 ppm, about 50 ppm, about 20 ppm, about 10 ppm, about 5 ppm, about 2 ppm, about 1 ppm, about 0.1 ppm, about 10 ppb, or about 1 ppb of chromium. In certain embodiments, the chromium-free sodium 1,2,3,4-tetrahydro-2-methyl-1,4-dioxo-2-naphthalenesulfonate contains no greater than about 100 ppm of chromium. In certain embodiments, the chromium-free sodium 1,2,3,4-tetrahydro-2-methyl-1,4-dioxo-2-naphthalenesulfonate contains no greater than about 50 ppm of chromium. In certain embodiments, the chromium-free sodium 1,2,3,4-tetrahydro-2-methyl-1,4-dioxo-2-naphthalenesulfonate contains no greater than about 20 ppm of chromium. In certain embodiments, the chromium-free sodium 1,2,3,4-tetrahydro-2-methyl-1,4-dioxo-2-naphthalenesulfonate contains no greater than about 10 ppm of chromium. In certain embodiments, the chromium-free sodium 1,2,3,4-tetrahydro-2-methyl-1,4-dioxo-2-naphthalenesulfonate contains no greater than about 5 ppm of chromium. In certain embodiments, the chromium-free sodium 1,2,3,4-tetrahydro-2-methyl-1,4-dioxo-2-naphthalenesulfonate contains no greater than about 2 ppm of chromium. In certain embodiments, the chromium-free sodium 1,2,3,4-tetrahydro-2-methyl-1,4-dioxo-2-naphthalenesulfonate contains no greater than about 1 ppm of chromium. In certain embodiments, the chromium-free sodium 1,2,3,4-tetrahydro-2-methyl-1,4-dioxo-2-naphthalenesulfonate contains no greater than about 0.1 ppm of chromium. In certain embodiments, the chromium-free sodium 1,2,3,4-tetrahydro-2-methyl-1,4-dioxo-2-naphthalenesulfonate contains no greater than about 10 ppb of chromium. In certain embodiments, the chromium-free sodium 1,2,3,4-tetrahydro-2-methyl-1,4-dioxo-2-naphthalenesulfonate contains no greater than about 1 ppb of chromium.

In certain embodiments, the chromium-free vitamin K₃ is made via a cerium mediator electrochemical technology (CETECH™) as described in U.S. Pat. No. 6,468,414, the disclosure of which is incorporated by reference herein in its entirety. Alternatively, chromium-free vitamin K₃ is available from commercial sources, such as PRO-K™.

In one embodiment, the quinone compound is one that is capable of increasing the production of a reactive oxygen species (e.g., in one embodiment superoxide anon, in another embodiment, hydrogen peroxide) in a cell.

In another embodiment, the quinone compound is one that is capable of inducing autoschizis. Taper et al., J. Histochem. Cytochem. 2001, 49, 109-19; Jamison et al., Biochem. Pharm. 2002, 63, 1773-83; the disclosure of each of which is incorporated herein by reference in its entirety.

In certain embodiments, the quinone compound is a naphthalenedione, optionally substituted with one or more substituents Q as defined herein. In certain embodiments, the quinone compound is a naphthalene-1,2-dione, optionally substituted with one or more substituents Q as defined herein. In certain embodiments, the quinone compound is a naphthalene-1,4-dione, optionally substituted with one or more substituents Q as defined herein. In certain embodiments, the quinone compound is a naphthalene-1,4-dione, substituted with one, two, three, or four substituents Q, each of which is independently selected from amino, halo, cyano, nitro, C₁₋₆ alkyl, —OR^{a}, —SR^{a}, and —COR^{a}, wherein R^{a} is (i) hydrogen; or (ii) C₁₋₆ alkyl, C₆₋₁₅ aryl, or heteroaryl, each optionally substituted with one or more substituents Q. In certain embodiments, the quinone compound is a naphthalene-1,4-dione, substituted with one, two, three, or four substituents Q, each of which is independently selected from amino, bromo, chloro, cyano, nitro, methyl, —OR^{a}, —SR^{a}, and —COR^{a}, wherein R^{a} is hydrogen, methyl, phenyl, chlorophenyl, fluorophenyl, tert-butylphenyl, methoxyphenyl, trimethoxyphenyl, or (methoxy-2-oxo-2H-chromenyl)methyl. In certain embodiments, the quinone compound is a naphthalene-1,4-dione, substituted with one, two, three, or four substituents Q, each of which is independently selected from amino, bromo, chloro, cyano, nitro, methyl, —OR^{a}, —SR^{a}, and —COR^{a}, wherein R^{a} is hydrogen, methyl, phenyl, 2-chlorophenyl, 3-chlorophenyl, 4-fluorophenyl, 4-tert-butylphenyl, 4-methoxyphenyl, 3,4,5-trimethoxyphenyl, or (7-methoxy-2-oxo-2H-chromen-4-yl)methyl. Additional quinone compounds include, but are not limited to, those disclosed in US 2013/0219528; and Benites et al. Invest. New Drugs 2011, 29, 760-7; the disclosure of each of which is incorporated herein by reference in its entirety.

In certain embodiments, the quinone compound is 2-bromo-1,4-naphthoquinone, 2-methoxy-1,4-naphthoquinone, or 2-methyl-1,4-naphthoquinone; or a pharmaceutically acceptable salt, solvate, hydrate, or prodrug thereof. In certain embodiments, the quinone compound is 2-(((7-methoxy-2-oxo-2H-chromen-4-yl)methyl)thio)naphthalene-1,4-dione, or a pharmaceutically acceptable salt, solvate, hydrate, or prodrug thereof. Additional quinone compounds include, but are not limited to, those disclosed in Bana et al., Mol. Carcinog. 2015, 54, 242-7, the disclosure of which is incorporated herein by reference in its entirety; or pharmaceutically acceptable salts, solvates, hydrates, and prodrugs thereof.

In certain embodiments, the quinone compound is 2-amino-3-bromo-1,4-naphthoquinone, 2-amino-3-chloro-1,4-naphthoquinone, or 2-amino-3-methoxy-1,4-naphthoquinone; or a pharmaceutically acceptable salt, solvate, hydrate, or prodrug thereof. In certain embodiments, the quinone compound is 2,3-dichloro-1,4-naphthoquinone or 2,3-dimethoxy-1,4-naphthoquinone; or a pharmaceutically acceptable salt, solvate, hydrate, or prodrug thereof. Additional quinone compounds include, but are not limited to, those disclosed in Graciani and Ximenes, Braz. J. Med. Biol. Res. 2012, 45, 701-10, the disclosure of which is incorporated herein by reference in its entirety; or pharmaceutically acceptable salts, solvates, hydrates, and prodrugs thereof.

In certain embodiments, the quinone compound is 2-dibenzoylamino-3-chloro-1,4-naphthoquinone, 2-dibenzoylamino-3-bromo-1,4-naphthoquinone, 2-dibenzoylamino-3-methoxy-1,4-naphthoquinone, 2-bis-(2-chlorobenzoyl)amino-3-chloro-1,4-naphthoquinone, 2-bis-(2-chlorobenzoyl)amino-3-bromo-1,4-naphthoquinone, 2-bis-(2-chlorobenzoyl)amino-3-methoxy-1,4-naphthoquinone, 2-bis-(3-chlorobenzoyl)amino-3-chloro-1,4-naphthoquinone, 2-bis-(3-chlorobenzoyl)amino-3-bromo-1,4-naphthoquinone, 2-bis-(3-chlorobenzoyl)amino-3-methoxy-1,4-naphthoquinone, 2-bis-(4-chlorobenzoyl)amino-3-chloro-1,4-naphthoquinone, 2-bis-(4-chlorobenzoyl)amino-3-bromo-1,4-naphthoquinone, 2-bis-(4-chlorobenzoyl)amino-3-methoxy-1,4-naphthoquinone, 2-bis-(4-fluorobenzoyl)amino-3-chloro-1,4-naphthoquinone, 2-bis-(4-fluorobenzoyl)amino-3-bromo-1,4-naphthoquinone, 2-bis-(4-fluorobenzoyl)amino-3-methoxy-1,4-naphthoquinone, 2-bis-(4-tert-butylbenzoyl)amino-3-chloro-1,4-naphthoquinone, 2-bis-(4-tert-butylbenzoyl)amino-3-bromo-1,4-naphthoquinone, 2-bis-(4-tert-butylbenzoyl)amino-3-methoxy-1,4-naphthoquinone, 2-bis-(4-methoxybenzoyl)amino-3-chloro-1,4-naphthoquinone, 2-bis-(4-methoxybenzoyl)amino-3-bromo-1,4-naphthoquinone, 2-bis-(3,4,5-trimethoxybenzoyl)-amino-3-chloro-1,4-naphthoquinone, 2-N-(4-chlorobenzoyl))-amino-3-chloro-1,4-naphthoquinone, 2-(N-benzoyl-N-(4-chlorobenzoyl))-amino-3-chloro-1,4-naphthoquinone, 2-N-acteylamino-3-chloro-1,4-naphthoquinone, or 2-(N-acetyl-N-(4-chlorobenzoyl))-amino-3-chloro-1,4-naphthoquinone; or a pharmaceutically acceptable salt, solvate, hydrate, or prodrug thereof. Additional quinone compounds include, but are not limited to, those disclosed in Brandy et al., Molecules 2013, 18, 1973-84, the disclosure of which is incorporated herein by reference in its entirety; or pharmaceutically acceptable salts, solvates, hydrates, and prodrugs thereof.

In certain embodiments, the quinone compound is plumbagin, also known as 5-hydroxy-2-methyl-naphthalene-1,4-dione. In certain embodiments, the quinone compound is plumbazeylanone. In certain embodiments, the quinone compound is lawsone, also known as 2-hydroxy-1,4-naphthoquinone. In certain embodiments, the quinone compound is juglone, also known as 5-hydroxy-1,4-naphthalenedione. Additional quinone compounds include, but are not limited to, those disclosed in Padhye et al., Med. Res. Rev. 2012, 32, 1131-58, the disclosure of which is incorporated herein by reference in its entirety; or pharmaceutically acceptable salts, solvates, hydrates, and prodrugs thereof.

In certain embodiments, the quinone compound is mitomycin C, also known as [6-amino-8a-methoxy-5-methyl-4,7-dioxo-1,1a,2,4,7,8,8a,8b-octahydroazireno[2′,3′:3,4]-pyrrolo[1,2-a]indol-8-yl]methyl carbamate. In certain embodiments, the quinone compound is daunorubicin, also known as (8S,10S)-8-acetyl-10-[(2S,4S,5S,6S)-4-amino-5-hydroxy-6-methyl-oxan-2-yl]oxy-6,8,11-trihydroxy-1-methoxy-9,10-dihydro-7H-tetracene-5,12-dione. In certain embodiments, the quinone compound is doxorubicin, also known as (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione. In certain embodiments, the quinone compound is mitoxantrone, also known as 1,4-dihydroxy-5,8-bis[2-(2-hydroxyethylamino)ethylamino]-anthracene-9,10-dione.

In certain embodiments, the quinone compound is chromium-free. In certain embodiments, the chromium-free quinone compound contains no more than about 100 ppm, about 50 ppm, about 20 ppm, about 10 ppm, about 5 ppm, about 2 ppm, about 1 ppm, about 0.1 ppm, about 10 ppb, or about 1 ppb of chromium. In certain embodiments, the chromium-free quinone compound contains no greater than about 100 ppm of chromium. In certain embodiments, the chromium-free quinone compound contains no greater than about 50 ppm of chromium. In certain embodiments, the chromium-free quinone compound contains no greater than about 20 ppm of chromium. In certain embodiments, the chromium-free quinone compound contains no greater than about 10 ppm of chromium. In certain embodiments, the chromium-free quinone compound contains no greater than about 5 ppm of chromium. In certain embodiments, the chromium-free quinone compound contains no greater than about 2 ppm of chromium. In certain embodiments, the chromium-free quinone compound contains no greater than about 1 ppm of chromium. In certain embodiments, the chromium-free quinone compound contains no greater than about 0.1 ppm of chromium. In certain embodiments, the chromium-free quinone compound contains no greater than about 10 ppb of chromium. In certain embodiments, the chromium-free quinone compound contains no greater than about 1 ppb of chromium.

The quinone compound may also be provided as a prodrug, which is a functional derivative of the quinone compound and is readily convertible into the parent quinone compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, a pharmaceutical composition for use in a method provided herein comprises (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound described herein, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof. In certain embodiments, the pharmaceutical composition further comprises a pharmaceutically acceptable excipient.

In one embodiment, the weight ratio of the ascorbic acid to the quinone compound in a pharmaceutical composition provided herein is ranging from about 4 to about 500, from about 10 to about 500, from about 50 to about 500, from about 25 to about 250, from about 50 to about 200, from about 50 to about 150, or from about 80 to about 120. In another embodiment, the weight ratio of the ascorbic acid to the quinone compound in a pharmaceutical composition provided herein is ranging from about 4 to about 500. In yet another embodiment, the weight ratio of the ascorbic acid to the quinone compound in a pharmaceutical composition provided herein is ranging from about 10 to about 500. In yet another embodiment, the weight ratio of the ascorbic acid to the quinone compound in a pharmaceutical composition provided herein is ranging from about 50 to about 500. In yet another embodiment, the weight ratio of the ascorbic acid to the quinone compound in a pharmaceutical composition provided herein is ranging from about 25 to about 250. In yet another embodiment, the weight ratio of the ascorbic acid to the quinone compound in a pharmaceutical composition provided herein is ranging from about 50 to about 200. In yet another embodiment, the weight ratio of the ascorbic acid to the quinone compound in a pharmaceutical composition provided herein is ranging from about 50 to about 150. In still another embodiment, the weight ratio of the ascorbic acid to the quinone compound in a pharmaceutical composition provided herein is ranging from about 80 to about 120.

In one embodiment, the weight ratio of the ascorbic acid to the quinone compound in a pharmaceutical composition provided herein is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250. In another embodiment, the weight ratio of the ascorbic acid to the quinone compound in a pharmaceutical composition provided herein is about 50. In yet another embodiment, the weight ratio of the ascorbic acid to the quinone compound in a pharmaceutical composition provided herein is about 100. In still another embodiment, the weight ratio of the ascorbic acid to the quinone compound in a pharmaceutical composition provided herein is about 200.

In one embodiment, the molar ratio of the ascorbic acid to the quinone compound in a pharmaceutical composition provided herein is ranging from about 10 to about 500, from about 25 to about 250, from about 50 to about 200, from about 50 to about 150, or from about 80 to about 120. In another embodiment, the molar ratio of the ascorbic acid to the quinone compound in a pharmaceutical composition provided herein is ranging from about 10 to about 500. In yet another embodiment, the molar ratio of the ascorbic acid to the quinone compound in a pharmaceutical composition provided herein is ranging from about 25 to about 250. In yet another embodiment, the molar ratio of the ascorbic acid to the quinone compound in a pharmaceutical composition provided herein is ranging from about 50 to about 200. In yet another embodiment, the molar ratio of the ascorbic acid to the quinone compound in a pharmaceutical composition provided herein is ranging from about 50 to about 150. In still another embodiment, the molar ratio of the ascorbic acid to the quinone compound in a pharmaceutical composition provided herein is ranging from about 80 to about 120.

In one embodiment, the molar ratio of the ascorbic acid to the quinone compound in a pharmaceutical composition provided herein is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250. In another embodiment, the molar ratio of the ascorbic acid to the quinone compound in a pharmaceutical composition provided herein is about 50. In yet another embodiment, the molar ratio of the ascorbic acid to the quinone compound in a pharmaceutical composition provided herein is about 100. In still another embodiment, the molar ratio of the ascorbic acid to the quinone compound in a pharmaceutical composition provided herein is about 200.

In certain embodiments, a pharmaceutical composition provided herein is formulated as a modified release dosage form, including, but not limited to, a delayed-, extended-, prolonged-, sustained-, pulsatile-, controlled-, accelerated-, fast-, targeted-, or programmed-release form. These dosage forms can be prepared according to conventional methods and techniques known to those skilled in the art (See, e.g., Remington: The Science and Practice of Pharmacy, supra; Modified-Release Drug Delivery Technology, Rathbone et al., Eds., Drugs and the Pharmaceutical Sciences, CRC Press LLC: 2008; Vol. 183).

In certain embodiments, a pharmaceutical composition provided herein is formulated for intratumoral administration. In certain embodiments, a pharmaceutical composition provided herein is formulated for intratumoral infusion. In certain embodiments, a pharmaceutical composition provided herein is formulated for intratumoral administration via direct injection. In certain embodiments, a pharmaceutical composition provided herein is formulated for intratumoral administration via implantation. In certain embodiments, a pharmaceutical composition provided herein is formulated for intratumoral administration via intracavitary delivery. In certain embodiments, a pharmaceutical composition provided herein is formulated for intratumoral administration via intracerebral delivery. In certain embodiments, a pharmaceutical composition provided herein is formulated for intratumoral administration via intracranial delivery. In certain embodiments, a pharmaceutical composition provided herein is formulated for intratumoral administration via intrathecal delivery. In certain embodiments, a pharmaceutical composition provided herein is formulated for intratumoral administration via intraventricular delivery. In certain embodiments, a pharmaceutical composition provided herein is formulated for intratumoral administration via convection-enhanced delivery (CED).

In one embodiment, the ascorbic acid in a pharmaceutical composition provided herein is L-ascorbic acid or a pharmaceutically acceptable salt thereof, or a pharmaceutically acceptable solvate or hydrate thereof. In another embodiment, the ascorbic acid in a pharmaceutical composition provided herein is an alkali or alkaline earth metal salt of L-ascorbic acid, or a pharmaceutically acceptable solvate or hydrate thereof; or a mixture thereof. In yet another embodiment, the ascorbic acid in a pharmaceutical composition provided herein is sodium, potassium, calcium, or magnesium salt of L-ascorbic acid, or a pharmaceutically acceptable solvate or hydrate thereof; or a mixture thereof. In yet another embodiment, the ascorbic acid in a pharmaceutical composition provided herein is sodium L-ascorbate. In yet another embodiment, the ascorbic acid in a pharmaceutical composition provided herein is calcium L-ascorbate. In yet another embodiment, the ascorbic acid in a pharmaceutical composition provided herein is magnesium L-ascorbate. In still another embodiment, the ascorbic acid in a pharmaceutical composition provided herein is a mixture of two or three of sodium L-ascorbate, calcium L-ascorbate, and magnesium L-ascorbate.

In one embodiment, the quinone compound in a pharmaceutical composition provided herein is vitamin K₃, or a pharmaceutically acceptable salt, solvate, or hydrate thereof. In another embodiment, the quinone compound in a pharmaceutical composition provided herein is 2-methyl-1,4-naphthalenedione, or a pharmaceutically solvate or hydrate thereof. In yet another embodiment, the quinone compound in a pharmaceutical composition provided herein is 2-methyl-1,4-naphthalenedione. In yet another embodiment, the quinone compound in a pharmaceutical composition provided herein is 1,2,3,4-tetrahydro-2-methyl-1,4-dioxo-2-naphthalenesulfonic acid, or a pharmaceutically acceptable salt, solvate, or hydrate thereof. In yet another embodiment, the quinone compound in a pharmaceutical composition provided herein is an alkali or alkaline earth metal salt of 1,2,3,4-tetrahydro-2-methyl-1,4-dioxo-2-naphthalenesulfonic acid, or a pharmaceutically acceptable solvate or hydrate thereof; or a mixture thereof. In yet another embodiment, the quinone compound in a pharmaceutical composition provided herein is sodium, potassium, calcium, or magnesium 1,2,3,4-tetrahydro-2-methyl-1,4-dioxo-2-naphthalenesulfonate, or a pharmaceutically acceptable solvate or hydrate thereof; or a mixture thereof. In yet another embodiment, the quinone compound in a pharmaceutical composition provided herein is sodium 1,2,3,4-tetrahydro-2-methyl-1,4-dioxo-2-naphthalenesulfonate, or a pharmaceutically acceptable solvate or hydrate thereof. In yet another embodiment, the quinone compound in a pharmaceutical composition provided herein is potassium 1,2,3,4-tetrahydro-2-methyl-1,4-dioxo-2-naphthalenesulfonate, or a pharmaceutically acceptable solvate or hydrate thereof. In yet another embodiment, the quinone compound in a pharmaceutical composition provided herein is magnesium 1,2,3,4-tetrahydro-2-methyl-1,4-dioxo-2-naphthalenesulfonate, or a pharmaceutically acceptable solvate or hydrate thereof. In yet another embodiment, the quinone compound in a pharmaceutical composition provided herein is sodium 1,2,3,4-tetrahydro-2-methyl-1,4-dioxo-2-naphthalenesulfonate. In yet another embodiment, the quinone compound in a pharmaceutical composition provided herein is anhydrous sodium 1,2,3,4-tetrahydro-2-methyl-1,4-dioxo-2-naphthalenesulfonate. In yet another embodiment, the quinone compound in a pharmaceutical composition provided herein is sodium 1,2,3,4-tetrahydro-2-methyl-1,4-dioxo-2-naphthalenesulfonate hydrate. In still another embodiment, the quinone compound in a pharmaceutical composition provided herein is sodium 1,2,3,4-tetrahydro-2-methyl-1,4-dioxo-2-naphthalenesulfonate trihydrate.

In certain embodiments, a pharmaceutical composition provided herein is provided in a unit-dosage or multiple-dosage form. A unit-dosage form, as used herein, refers to a physically discrete unit suitable for administration to a subject, e.g., a human and animal subject, and packaged individually as is known in the art. Each unit-dose contains a predetermined quantity of one or more active ingredient(s) sufficient to produce the desired therapeutic effect, optionally in association with one or more pharmaceutical vehicle(s), carrier(s), diluent(s), or excipient(s). Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form.

A pharmaceutical composition provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Methods of Use

In one embodiment, provided herein is a method of treating or alleviating one or more symptoms of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

In another embodiment, provided herein is a method of inhibiting the growth of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

In yet another embodiment, provided herein is a method of increasing the quality of life of a subject having a tumor, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

In certain embodiments, the tumor is a solid tumor. In certain embodiments, the tumor is a resectable tumor. In certain embodiments, the tumor is an unresectable tumor. In certain embodiments, the tumor is a recurrent tumor.

In certain embodiments, the tumor is a benign tumor. In certain embodiments, the tumor is a resectable benign tumor. In certain embodiments, the tumor is an unresectable benign tumor. In certain embodiments, the tumor is a recurrent benign tumor.

In certain embodiments, the tumor is a malignant tumor. In certain embodiments, the tumor is a resectable malignant tumor. In certain embodiments, the tumor is an unresectable malignant tumor. In certain embodiments, the tumor is a recurrent malignant tumor.

In certain embodiments, the malignant tumor is refractory and/or relapsed. In certain embodiments, the malignant tumor is refractory. In certain embodiments, the malignant tumor is relapsed. In certain embodiments, the malignant tumor is metastatic. In certain embodiments, the malignant tumor is drug-resistant. In certain embodiments, the malignant tumor is multidrug-resistant.

In certain embodiments, the tumor is a brain tumor. In certain embodiments, the tumor is a benign brain tumor. In certain embodiments, the brain tumor is resectable. In certain embodiments, the brain tumor is unresectable. In certain embodiments, the brain tumor is recurrent.

In certain embodiments, the brain tumor is a malignant brain tumor. In certain embodiments, the malignant brain tumor is resectable. In certain embodiments, the malignant brain tumor is unresectable. In certain embodiments, the malignant brain tumor is recurrent.

In certain embodiments, the malignant brain tumor is refractory and/or relapsed. In certain embodiments, the malignant brain tumor is refractory. In certain embodiments, the malignant brain tumor is relapsed. In certain embodiments, the malignant brain tumor is metastatic. In certain embodiments, the malignant brain tumor is drug-resistant. In certain embodiments, the malignant brain tumor is multidrug-resistant.

In certain embodiments, the brain tumor is a primary brain tumor. In certain embodiments, the brain tumor is a secondary or metastatic brain tumor.

In certain embodiments, the brain tumor is a glioma. In certain embodiments, the brain tumor is a benign glioma. In certain embodiments, the benign glioma is resectable. In certain embodiments, the benign glioma is unresectable. In certain embodiments, the benign glioma is recurrent.

In certain embodiments, the glioma is a malignant glioma. In certain embodiments, the malignant glioma is resectable. In certain embodiments, the malignant glioma is unresectable. In certain embodiments, the malignant glioma is recurrent.

In certain embodiments, the malignant glioma is refractory and/or relapsed. In certain embodiments, the malignant glioma is refractory. In certain embodiments, the malignant glioma is relapsed. In certain embodiments, the malignant glioma is metastatic. In certain embodiments, the malignant glioma is drug-resistant. In certain embodiments, the malignant glioma is multidrug-resistant.

In certain embodiments, the brain tumor is anaplastic astrocytoma, anaplastic ependymoma, anaplastic oligodendroglioma, craniopharyngioma, diffuse astrocytoma, ependymoblastoma, gangliocytoma, ganglioglioma, glioblastoma, medulloblastoma, pineoblastoma, pineocytoma, pilocytic astrocytoma, or pure oligodendroglioma. In certain embodiments, the brain tumor is astrocytoma, glioblastoma or glioblastoma multiforme (GBM), meningioma, or medulloblastoma.

In certain embodiments, the brain tumor is astrocytoma. In certain embodiments, astrocytoma is resectable. In certain embodiments, astrocytoma is unresectable. In certain embodiments, astrocytoma is recurrent. In certain embodiments, astrocytoma is refractory and/or relapsed. In certain embodiments, astrocytoma is refractory. In certain embodiments, astrocytoma is relapsed. In certain embodiments, astrocytoma is metastatic. In certain embodiments, astrocytoma is drug-resistant. In certain embodiments, astrocytoma is multidrug-resistant.

In certain embodiments, the brain tumor is glioblastoma. In certain embodiments, glioblastoma is resectable. In certain embodiments, glioblastoma is unresectable. In certain embodiments, glioblastoma is recurrent. In certain embodiments, glioblastoma is refractory and/or relapsed. In certain embodiments, glioblastoma is refractory. In certain embodiments, glioblastoma is relapsed. In certain embodiments, glioblastoma is metastatic. In certain embodiments, glioblastoma is drug-resistant. In certain embodiments, glioblastoma is multidrug-resistant.

In certain embodiments, the brain tumor is meningioma. In certain embodiments, meningioma is resectable. In certain embodiments, meningioma is unresectable. In certain embodiments, meningioma is recurrent. In certain embodiments, meningioma is refractory and/or relapsed. In certain embodiments, meningioma is refractory. In certain embodiments, meningioma is relapsed. In certain embodiments, meningioma is metastatic. In certain embodiments, meningioma is drug-resistant. In certain embodiments, meningioma is multidrug-resistant.

In certain embodiments, the brain tumor is medulloblastoma. In certain embodiments, medulloblastoma is resectable. In certain embodiments, medulloblastoma is unresectable. In certain embodiments, medulloblastoma is recurrent. In certain embodiments, medulloblastoma is refractory and/or relapsed. In certain embodiments, medulloblastoma is refractory. In certain embodiments, medulloblastoma is relapsed. In certain embodiments, medulloblastoma is metastatic. In certain embodiments, medulloblastoma is drug-resistant. In certain embodiments, medulloblastoma is multidrug-resistant.

In certain embodiments, the ascorbic acid and quinone compound as used in a method provided herein are delivered as a single dose, such as, e.g., as a single bolus injection. In certain embodiments, the ascorbic acid and quinone compound as used in a method provided herein are administered over time, such as, e.g., continuous infusion over time or divided bolus doses over time.

In certain embodiments, the weight ratio of the ascorbic acid to the quinone compound as used in a method provided herein is ranging from about 1 to about 1,000, from about 10 to about 500, from about 50 to about 500, from about 100 to about 500, from about 200 to about 500, or from about 200 to about 400. In certain embodiments, the weight ratio of the ascorbic acid to the quinone compound as used in a method provided herein is ranging from about 1 to about 1,000. In certain embodiments, the weight ratio of the ascorbic acid to the quinone compound as used in a method provided herein is ranging from about 10 to about 500. In certain embodiments, the weight ratio of the ascorbic acid to the quinone compound as used in a method provided herein is ranging from about 50 to about 500. In certain embodiments, the weight ratio of the ascorbic acid to the quinone compound as used in a method provided herein is ranging from about 100 to about 500. In certain embodiments, the weight ratio of the ascorbic acid to the quinone compound as used in a method provided herein is ranging from about 200 to about 500. In certain embodiments, the weight ratio of the ascorbic acid to the quinone compound as used in a method provided herein is ranging from about 200 to about 400. In certain embodiments, the weight ratio of the ascorbic acid to the quinone compound as used in a method provided herein is about 100, about 200, about 300, about 400, about 500, or about 600. In certain embodiments, the weight ratio of the ascorbic acid to the quinone compound as used in a method provided herein is about 200. In certain embodiments, the weight ratio of the ascorbic acid to the quinone compound as used in a method provided herein is about 400.

In certain embodiments, the weight ratio of the ascorbic acid to the quinone compound as used in a method provided herein is ranging from about 25 to about 250, from about 50 to about 200, from about 50 to about 150, or from about 80 to about 120. In certain embodiments, the weight ratio of the ascorbic acid to the quinone compound as used in a method provided herein is ranging from about 25 to about 250. In certain embodiments, the weight ratio of the ascorbic acid to the quinone compound as used in a method provided herein is ranging from about 50 to about 200. In certain embodiments, the weight ratio of the ascorbic acid to the quinone compound as used in a method provided herein is ranging from about 50 to about 150. In certain embodiments, the weight ratio of the ascorbic acid to the quinone compound as used in a method provided herein is ranging from about 80 to about 120. In certain embodiments, the weight ratio of the ascorbic acid to the quinone compound as used in a method provided herein is about 1, about 2, about 4, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250. In certain embodiments, the weight ratio of the ascorbic acid to the quinone compound as used in a method provided herein is about 50. In certain embodiments, the weight ratio of the ascorbic acid to the quinone compound as used in a method provided herein is about 100.

In certain embodiments, the ascorbic acid as used in a method provided herein is administered to the subject in an amount ranging from about 0.1 to about 500 mg/kg/day, from about 0.2 to about 200 mg/kg/day, from about 0.5 to about 100 mg/kg/day, or from about 1 to about 100 mg/kg/day. In certain embodiments, the ascorbic acid as used in a method provided herein is administered to the subject in an amount ranging from about 0.1 to about 500 mg/kg/day. In certain embodiments, the ascorbic acid as used in a method provided herein is administered to the subject in an amount ranging from about 0.2 to about 200 mg/kg/day. In certain embodiments, the ascorbic acid as used in a method provided herein is administered to the subject in an amount ranging from about 0.5 to about 100 mg/kg/day. In certain embodiments, the ascorbic acid as used in a method provided herein is administered to the subject in an amount ranging from about 1 to about 100 mg/kg/day. In certain embodiments, the ascorbic acid as used in a method provided herein is administered to the subject in an amount of about 0.5 mg/kg/day, about 1 mg/kg/day, about 2 mg/kg/day, about 5 mg/kg/day, about 10 mg/kg/day, or about 20 mg/kg/day.

In certain embodiments, the ascorbic acid as used in a method provided herein is administered to the subject in an amount ranging from about 0.1 to about 500 mg/kg per treatment, from about 0.2 to about 200 mg/kg per treatment, from about 0.5 to about 100 mg/kg per treatment, or from about 1 to about 100 mg/kg per treatment. In certain embodiments, the ascorbic acid as used in a method provided herein is administered to the subject in an amount ranging from about 0.1 to about 500 mg/kg per treatment. In certain embodiments, the ascorbic acid as used in a method provided herein is administered to the subject in an amount ranging from about 0.2 to about 200 mg/kg per treatment. In certain embodiments, the ascorbic acid as used in a method provided herein is administered to the subject in an amount ranging from about 0.5 to about 100 mg/kg per treatment. In certain embodiments, the ascorbic acid as used in a method provided herein is administered to the subject in an amount ranging from about 1 to about 100 mg/kg per treatment. In certain embodiments, the ascorbic acid as used in a method provided herein is administered to the subject in an amount of about 0.5 mg/kg per treatment, about 1 mg/kg per treatment, about 2 mg/kg per treatment, about 5 mg/kg per treatment, about 10 mg/kg per treatment, or about 20 mg/kg per treatment.

In certain embodiments, the quinone compound as used in a method provided herein is administered to the subject in an amount ranging from about 0.001 to about 5 mg/kg/day, from about 0.002 to about 2 mg/kg/day, from about 0.005 to about 1 mg/kg/day, or from about 0.01 to about 1 mg/kg/day. In certain embodiments, the quinone compound as used in a method provided herein is administered to the subject in an amount ranging from about 0.001 to about 5 mg/kg/day. In certain embodiments, the quinone compound as used in a method provided herein is administered to the subject in an amount ranging from about 0.002 to about 2 mg/kg/day. In certain embodiments, the quinone compound as used in a method provided herein is administered to the subject in an amount ranging from about 0.005 to about 1 mg/kg/day. In certain embodiments, the quinone compound as used in a method provided herein is administered to the subject in an amount ranging from about 0.01 to about 1 mg/kg/day. In certain embodiments, the quinone compound as used in a method provided herein is administered to the subject in an amount of about 0.01 mg/kg/day, about 0.02 mg/kg/day, about 0.05 mg/kg/day, or about 0.1 mg/kg/day.

In certain embodiments, the quinone compound as used in a method provided herein is administered to the subject in an amount ranging from about 0.001 to about 5 mg/kg per treatment, from about 0.002 to about 2 mg/kg per treatment, from about 0.005 to about 1 mg/kg per treatment, or from about 0.01 to about 1 mg/kg per treatment. In certain embodiments, the quinone compound as used in a method provided herein is administered to the subject in an amount ranging from about 0.001 to about 5 mg/kg per treatment. In certain embodiments, the quinone compound as used in a method provided herein is administered to the subject in an amount ranging from about 0.002 to about 2 mg/kg per treatment. In certain embodiments, the quinone compound as used in a method provided herein is administered to the subject in an amount ranging from about 0.005 to about 1 mg/kg per treatment. In certain embodiments, the quinone compound as used in a method provided herein is administered to the subject in an amount ranging from about 0.01 to about 1 mg/kg per treatment. In certain embodiments, the quinone compound as used in a method provided herein is administered to the subject in an amount of about 0.01 mg/kg per treatment, about 0.02 mg/kg per treatment, about 0.05 mg/kg per treatment, or about 0.1 mg/kg per treatment.

The administered doses of the ascorbic acid and quinone compound can also each independently be expressed in units other than the unit “mg/kg/day” or “g/kg/day.” For example, doses for parenteral administration can be expressed as mg/m²/day. One of ordinary skill in the art would readily know how to convert doses from mg/kg/day to mg/m²/day, given either the height or weight of a subject or both. For example, a dose of 1 mg/kg/day for a 65 kg human is approximately equal to 38 mg/m²/day.

In certain embodiments, the ascorbic acid used in a method provided herein is administered intratumorally. In certain embodiments, the ascorbic acid used in a method provided herein is administered by intratumoral infusion. In certain embodiments, the ascorbic acid used in a method provided herein is administered intratumorally via direct injection. In certain embodiments, the ascorbic acid used in a method provided herein is administered intratumorally via intracavitary delivery. In certain embodiments, the ascorbic acid used in a method provided herein is administered intratumorally via intracerebral delivery. In certain embodiments, the ascorbic acid used in a method provided herein is administered intratumorally via intracranial delivery. In certain embodiments, the ascorbic acid used in a method provided herein is administered intratumorally via intrathecal delivery. In certain embodiments, the ascorbic acid used in a method provided herein is administered intratumorally via intraventricular delivery. In certain embodiments, the ascorbic acid used in a method provided herein is administered intratumorally via convection-enhanced delivery (CED).

In certain embodiments, the quinone compound used in a method provided herein is administered intratumorally. In certain embodiments, the quinone compound used in a method provided herein is administered by intratumoral infusion. In certain embodiments, the quinone compound used in a method provided herein is administered intratumorally via direct injection. In certain embodiments, the quinone compound used in a method provided herein is administered intratumorally via intracavitary delivery. In certain embodiments, the quinone compound used in a method provided herein is administered intratumorally via intracerebral delivery. In certain embodiments, the quinone compound used in a method provided herein is administered intratumorally via intracranial delivery. In certain embodiments, the quinone compound used in a method provided herein is administered intratumorally via intrathecal delivery. In certain embodiments, the quinone compound used in a method provided herein is administered intratumorally via intraventricular delivery. In certain embodiments, the quinone compound used in a method provided herein is administered intratumorally via CED.

In certain embodiments, the ascorbic acid and quinone compound used in a method provided herein are administered together intratumorally. In certain embodiments, the ascorbic acid and quinone compound used in a method provided herein are administered by intratumoral infusion. In certain embodiments, the ascorbic acid and quinone compound used in a method provided herein are administered together intratumorally via direct injection. In certain embodiments, the ascorbic acid and quinone compound used in a method provided herein are administered together intratumorally via implantation. In certain embodiments, the ascorbic acid and quinone compound used in a method provided herein are administered together intratumorally via intracavitary delivery. In certain embodiments, the ascorbic acid and quinone compound used in a method provided herein are administered together intratumorally via intracerebral delivery. In certain embodiments, the ascorbic acid and quinone compound used in a method provided herein are administered together intratumorally via intracranial delivery. In certain embodiments, the ascorbic acid and quinone compound used in a method provided herein are administered together intratumorally via intrathecal delivery. In certain embodiments, the ascorbic acid and quinone compound used in a method provided herein are administered together intratumorally via intraventricular delivery. In certain embodiments, the ascorbic acid and quinone compound used in a method provided herein are administered together intratumorally via CED.

In certain embodiments, the ascorbic acid and quinone compound as used in a method provided herein are administered once a month, once every two weeks, once a week, or once daily (QD). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

In certain embodiments, the ascorbic acid and quinone compound as used in a method provided herein are cyclically administered to a subject. Cycling therapy involves the administration of active agent(s) for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

In certain embodiments, the ascorbic acid and quinone compound are administered repetitively, if necessary, for example, until the subject experiences stable disease or regression, or until the subject experiences disease progression or unacceptable toxicity. Stable disease or lack thereof is determined by a method known in the art such as evaluation of subject's symptoms, physical examination, or diagnostic testing.

The ascorbic acid and quinone compound as used in a method provided herein can also be combined or used in combination with a second therapeutic agent useful in the treatment of the tumor described herein. In certain embodiments, the second therapeutic agent is an anticancer agent. In certain embodiments, the second therapeutic agent is bevacizumab, carmustine, everolimus, lomustine, or temozolomide. In certain embodiments, the second therapeutic agent is a farnesyltransferase inhibitor. In certain embodiments, the farnesyltransferase inhibitor is gingerol, gliotoxin, lonafarnib, tipifarnib, FTI-277, GGTI-287, GGTI-2418, or L-778123. In certain embodiments, the farnesyltransferase inhibitor is lonafarnib or tipifarnib.

In certain embodiments, the methods provided herein encompass treating a subject regardless of patient's age. In certain embodiments, the subject is a pediatric patient. In certain embodiments, the subject is an adult patient.

In certain embodiments, the subject is a mammal. In certain embodiments, the subject is a human.

In one embodiment, provided herein is a method of inhibiting the growth of a tumorous cell, comprising contacting the tumorous cell with an effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

In certain embodiments, the tumorous cell is a benign tumorous cell. In certain embodiments, the tumorous cell is a human benign tumorous cell. In certain embodiments, the tumorous cell is a malignant tumorous cell. In certain embodiments, the tumorous cell is a human malignant tumorous cell.

In certain embodiments, the tumorous cell is a glioma cell. In certain embodiments, the tumorous cell is a benign glioma cell. In certain embodiments, the tumorous cell is a malignant glioma cell. In certain embodiments, the tumorous cell is a human malignant glioma cell. In certain embodiments, the tumorous cell is a glioblastoma cell. In certain embodiments, the tumorous cell is a human malignant glioblastoma cell.

In certain embodiments, provided herein are kits which, when used by the medical practitioner, can simplify the administration of appropriate amounts of active ingredients to a subject. In certain embodiments, the kit provided herein includes containers and dosage forms of the active ingredients provided herein. Kits provided herein can further include devices that are used to administer the active ingredients.

The disclosure will be further understood by the following non-limiting examples.

### EXAMPLES

As used herein, the symbols and conventions used in the examples, regardless of whether a particular abbreviation is specifically defined, are consistent with those used in the contemporary scientific literature, for example, the Journal of the American Chemical Society, the Journal of Medicinal Chemistry. or the Journal of Biological Chemistry. Specifically, but without limitation, the following abbreviations may be used in the examples and throughout the specification: kg (kilograms); g (grams); mg (milligrams); μg (micrograms); L (liters); mL (milliliters); μL (microliters); mM (millimolar); μM (micromolar); mmol (millimoles); h (hour or hours); min (minute or minutes); DMSO (dimethylsulfoxide); and MRI (magnetic resonance imaging).

### Example 1

### Antitumor Activity of Vitamins C and Menadione in a Xenograft Mouse Model

Six-week-old male Balb/c nude mice were maintained in specific pathogen-free conditions. Glioblastoma cells (GS9L) were cultured in DMEM supplemented with 10% FBS, penicillin (100 U/mL), and streptomycin (100 mg/L) at 37° C. in a humidified atmosphere containing 5% CO₂. After deep anesthesia, the mice were positioned in a stereotactic frame. A small craniectomy was performed at 2-3 mm from the midline and 1 mm anterior to the coronal suture. The glioblastoma cells (5×10⁵ cells in 5 μL) were stereotactically injected into the brain parenchyma at a depth of 3 mm on Day 1 using an automated injector.

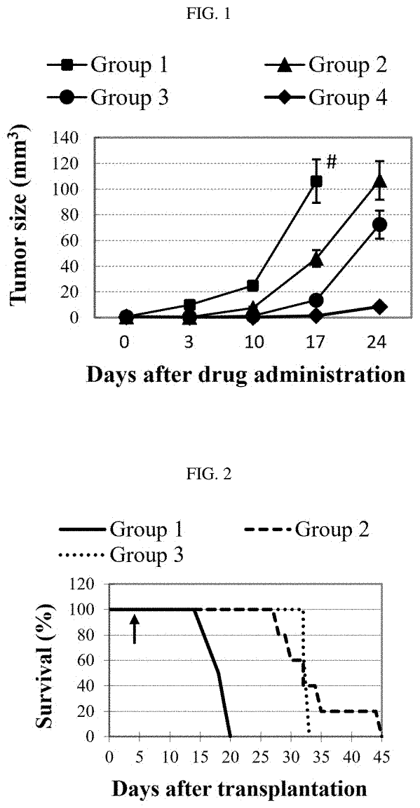

Vitamin C (L-ascorbic acid) was dissolved in PBS at 10 mM (pH 7.4). Menadione and temozolomide (TMZ) were each dissolved in DMSO at 10 mM as a stock solution and diluted in PBS when in use. The final concentration of DMSO was below 1%. On Day 4, the mice were treated via intracranial injection with (i) a saline solution (10 μL) in Group 1 of 5 mice as a control; (ii) a solution (10 μL) containing L-ascorbic acid (1.4 mg/kg) and menadione (14 μg/kg) in Group 2 of 3 mice; (iii) a solution (10 μL) containing L-ascorbic acid (7 mg/kg) and menadione (70 μg/kg) in Group 3 of 4 mice; or (iv) a solution (10 μL) containing TMZ (4 mg/kg) in Group 4 of 5 mice. After the intracranial treatment, the glioblastoma-bearing mice in the control group and the TMZ-treatment group were fed with regular water and the glioblastoma-bearing mice in the treatment groups were fed orally with water containing L-ascorbic acid (15 g/L) and menadione (150 mg/L) daily. The glioblastoma-bearing mice were analyzed on Days 4, 7, 14, 21, and 28 (i.e., 0, 3, 10, 17, and 24 days after drug administration) by MRI to determine the size of the glioblastoma. In the control group, the last measurement of tumor size by MRI was 15 or 17 days after drug administration, depending on health condition and endpoint. The survival of the glioblastoma-bearing mice was also followed. The results are summarized in FIGS. 1 and 2. All values in the treatment groups were statistically significant compared to the control group. The mice in the TMZ treatment group experienced side-effects (e.g., fever, tetraplegia, or convulsions), whereas the mice in the vitamin C/menadione treatment groups did not.

### Example 2

### Antitumor Activity of Vitamins C and K₃ in a Xenograft Mouse Model

Six-week-old male Balb/c nude mice were maintained in specific pathogen-free conditions. Glioblastoma cells (U-87 MG) were cultured in DMEM supplemented with 10% FBS, penicillin (100 U/mL), and streptomycin (100 mg/L) at 37° C. in a humidified atmosphere containing 5% CO₂. After deep anesthesia, the mice were positioned in a stereotactic frame. A small craniectomy was performed at 2-3 mm from the midline and 1 mm anterior to the coronal suture. The glioblastoma cells (3×10⁵ cells in 5 μL) were stereotactically injected into the brain parenchyma at a depth of 3 mm on Day 1 using an automated injector.

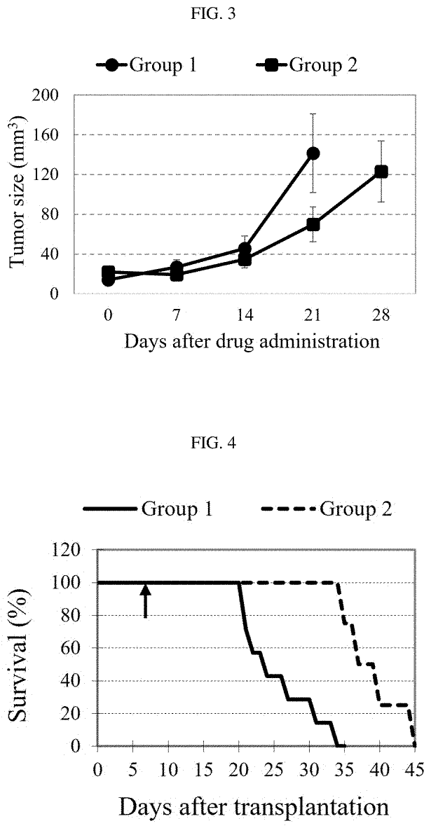

Vitamin C (L-ascorbic acid) was dissolved in PBS at 10 mM (pH 7.4). Menadione was dissolved in DMSO at 10 mM as a stock solution and diluted in PBS when in use. The final concentration of DMSO was below 1%. On Day 7, the mice were treated intratumorally via intracranial injection with (i) a saline solution (5 μL) in Group 1 of 7 mice as a control; or (ii) a solution μL containing L-ascorbic acid (7 mg/kg) and menadione (70 μg/kg) in Group 2 of 5 mice. After the intracranial treatment, the glioblastoma-bearing mice in the control group were fed with regular water and the glioblastoma-bearing mice in the treatment group were fed orally with water containing L-ascorbic acid (15 g/L) and menadione (150 mg/L) daily. The glioblastoma-bearing mice were analyzed on Days 7, 14, 21, 28, and 35 (i.e., 0, 7, 14, 21, and 28 days after drug administration) by MRI to determine the size of the glioblastoma. The survival of the glioblastoma-bearing mice was also followed. The results are summarized in FIGS. 3 and 4.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method of treating or alleviating one or more symptoms of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

2. A method of inhibiting the growth of a tumor in a subject, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

3. A method of increasing the quality of life of a subject having a tumor, comprising administering intratumorally to the subject in need thereof a therapeutically effective amount of: (i) ascorbic acid, or a diastereomer, a mixture of two or more diastereomers, a tautomer, a mixture of two or more tautomers; or a pharmaceutically acceptable salt, solvate, or hydrate thereof; and (ii) a quinone compound, or an enantiomer, a mixture of enantiomers, a diastereomer, a mixture of two or more diastereomers, a tautomer, or a mixture of two or more tautomers; or a pharmaceutically acceptable salt, solvate, hydrate, or prodrug thereof.

4. The method of claim 1, wherein the tumor is a solid tumor.

5. The method of claim 1, wherein the tumor is a benign tumor.

6. The method of claim 1, wherein the tumor is a malignant tumor.

7. The method of claim 1, wherein the tumor is a brain tumor, a primary brain tumor, or a secondary brain tumor.

8-9. (canceled)

10. The method of claim 1, wherein the tumor is anaplastic astrocytoma, anaplastic ependymoma, anaplastic oligodendroglioma, craniopharyngioma, diffuse astrocytoma, ependymoblastoma, gangliocytoma, ganglioglioma, glioblastoma, medulloblastoma, pineoblastoma, pineocytoma, pilocytic astrocytoma, or pure oligodendroglioma.

11. The method of claim 1, wherein the tumor is a glioma.

12. (canceled)

13. The method of claim 1, wherein the tumor is glioblastoma.

14-19. (canceled)

20. The method of claim 1, wherein the ascorbic acid is L-ascorbic acid or a pharmaceutically acceptable salt thereof, or a pharmaceutically acceptable solvate or hydrate thereof.

21-27. (canceled)

28. The method of claim 1, wherein the quinone compound is vitamin K.

29. The method of claim 1, wherein the quinone compound is vitamin K₃.

30. The method of claim 29, wherein vitamin K₃ is 1,2,3,4-tetrahydro-2-methyl-1,4-dioxo-2-naphthalenesulfonic acid or a pharmaceutically acceptable salt thereof; or a pharmaceutically acceptable solvate or hydrate thereof.

31-33. (canceled)

34. The method of claim 29, wherein vitamin K₃ is 2-methyl-1,4-naphthalenedione, or a pharmaceutically acceptable solvate or hydrate thereof.

35. The method of claim 1, wherein the weight ratio of the ascorbic acid to the quinone compound is ranging from about 10 to about 500.

36. (canceled)

37. The method of claim 1, wherein the ascorbic acid is administered via direction injection, intracerebral delivery, intracranial delivery, convection-enhanced delivery, or implantation, or subcutaneously.

38-42. (canceled)

43. The method of claim 1, wherein the quinone compound is administered via direction injection, intracerebral delivery, intracranial delivery, convection-enhanced delivery, or implantation, or subcutaneously.

44-48. (canceled)

49. The method of claim 1, wherein the ascorbic acid and quinone compound are administered via direction injection, intracerebral delivery, intracranial delivery, convection-enhanced delivery, or implantation, or subcutaneously.

50-54. (canceled)

55. The method of claim 1, wherein the ascorbic acid and the quinone compound are administered together.

56-59. (canceled)
